(19) United States
(12) Patent Application Publication
(10) Pub. No.: US 2020/0138446 A1
(43) Pub. Date: May 7, 2020

(54) IMPLANT FOR INSERTION INTO A BODY CAVITY

(71) Applicant: RHEINISCH-WESTFALISCHE TECH-NISCHE HOCHSCHULE AACHEN, Aachen (DE)

(72) Inventors: Stefan JOCKENHOEVEL, Aachen (DE); Hermann MONSTADT, Bochum (DE)

(21) Appl. No.: 16/609,352

(22) PCT Filed: May 17, 2018
(86) PCT No.: PCT/EP2018/000286

§ 371 Date: Oct. 29, 2019

(30) Foreign Application Priority Data
May 17, 2017 (DE) 10 2017 004 700.6

(51) Int. Cl. A61B 17/12

(52) U.S. Cl. CPC A61B 17/12113; A61B 17/12145; A61B 2017/00004; A61B 17/12031; A61B 17/1215

(57) ABSTRACT

Implant for insertion into body cavities, in particular blood vessels, preferably aneurysms, comprising at least one fibre (2) by means of which a coagulation of blood can be brought about, wherein the implant comprises at least one driving element (1) which can be formed into a first shape and which can be converted from the first shape automatically into a second, in particular relaxed shape by a mechanical stress which is applied to the element in the first shape, and the implant comprises at least one driven fibre (2), in particular a multiplicity of driven fibres (2), and a respective driven fibre (2) with regions which are spaced apart in the direction of extent of the fibre is attached to the at least one driving element (1) in regions which are at a different position with respect to one another, in particular at a larger distance from one another, in the first shape than in the second shape, and the at least one driven fibre (2), preferably the multiplicity of driven fibres (2), is driven by the driving element (1) to change its shape with the conversion of the driving element (1) from the first shape into the second shape.

The invention relates to an implant for insertion into body cavities, in particular for insertion into blood vessels and preferably into aneurysms, comprising at least one fiber, preferably a plurality of fibers, in particular by means of which a coagulation of blood can be brought about.

Implants of this type are known in the prior art and are used e.g. in order to close off body cavities, preferably aneurysms, permanently. The effect of these implants is based on the fact that the fibers cause the blood flow in the body cavity to slow down and as a result thereof bring about thrombosis of the cavity. As a result, a body cavity, preferably an aneurysm, can be closed off by the blood clot which is formed and which develops around the implant.

Implants which are known in the prior art comprise fibers which, through their mere presence or through a coating, promote formation of thrombi. For example, such fibers can be attached individually or as a bundle to twisted core wires in such a way that the fibers are oriented essentially perpendicularly with respect to the direction of the core wire, that is to say protrude radially from the twisted core wires. Such an implant is disclosed by e.g. DE 10 2007 025 466.

Such an implant can be pushed by means of a guide aid, e.g. a guide wire, through a catheter into the body cavity to be treated and disconnected from the guide aid there, with the result that it remains in the body cavity.

It is a problem with such implants that they have, in the direction of extent of the twisted core wires, a degree of flexural rigidity which counteracts a compressibility of the implant in this direction. Depending on the application, the insertion of such an implant into a body cavity thus proves difficult, in particular if in order to achieve a high filling level a plurality of such implants are to be inserted into the body cavity, and the implants are therefore to be plugged e.g. successively into the cavity.

An object of the invention is therefore to make available an implant of the generic type mentioned at the beginning which permits not only good guidability in a catheter but also allows a high filling level in a body cavity to be achieved, in particular if a plurality of such implants are to be inserted into the body cavity, e.g. an aneurysm.

According to the invention, this object is achieved by means of an implant of the generic type mentioned at the beginning which comprises at least one driving element, or comprises precisely one driving element, which can be formed into a first shape and which can be converted from the first shape automatically into a second, in particular relaxed shape by a mechanical stress which is applied to the element in the first shape, and the implant comprises at least one driven fiber, in particular a multiplicity of driven fibers, and a respective driven fiber with regions which are spaced apart in the direction of extent of the fiber is attached to the at least one driving element in regions which are at a different position with respect to one another, in particular at a larger distance from one another, in the first shape than in the second shape, and the at least one driven fiber, preferably the multiplicity of driven fibers, is driven by the driving element to change its shape with the conversion of the driving element from the first shape into the second shape.

The essential core idea of the invention is that the implant can change its shape automatically or changes during or after the positioning in the body cavity, wherein it is preferably provided that, in the first shape of the driving element, the implant overall has a shape which is such that it promotes guidance of the implant in a catheter. For example, in the specified first shape of the driving element, the implant can have a shape which is such that the implant is elongated in a predefined direction, in particular which corresponds to the direction of extent of the catheter, in particular in this context has the largest extent in this direction of longitudinal extent and the smallest extent perpendicularly with respect thereto.

In this first shape, the specified mechanical stress is impressed on the at least one driving element of the implant, which stress relieves the implant automatically, e.g. after the release from the catheter, that is to say changes into a relaxed state of the driving element. The relaxation can occur directly as a result of the release from the catheter or else as a result of an external induction, that is to say an external effect.

A system composed of a catheter and an implant can therefore be embodied e.g. in one embodiment in such a way that the catheter holds the implant in the first specified shape of the at least one driving element, and the element automatically changes into the second shape when the effect of the catheter on the implant is eliminated. In another embodiment, in the first shape of the at least one driving element the implant can also be intrinsically stable and require the specified induction through an external event, e.g. warming by the blood, in order to change into the second shape of the element and therefore overall carry out a change in shape.

The change in shape of the implant overall is generated essentially by virtue of the fact that the at least one driven fiber also carries out a change in shape by way of the change in shape of the at least one driving element from the first into the second shape because the at least one driving element applies a shape-changing force to the at least one driven fiber. Therefore, each driven fiber also changes from a first into a second shape when the at least one driving element carries out the change in shape. The specific shapes of a driving element or elements and a driven fiber or fibers are not necessarily the same here, in particular they are preferably different, i.e. the change in shape of the at least one driving element is different to that of the driven fiber or fibers. In particular, the change in shape of the at least one driving element is precisely predetermined, i.e. the first and second shape of the at least one or of the precisely one driving element are defined, while the change in shape of the at least one fiber is dependent on the change in shape of the driving element but indefinite. It is to be generally noted here that a feature which is described with respect to at least one driven fiber or one driving element can, given the presence of a plurality of fibers or elements, accordingly also be respectively implemented for all these fibers or elements.

Irrespective of the type of triggering of the change in shape which is caused by the at least one driving element of the implant, the change in shape preferably brings about a shortening of the implant with respect to the first shape and therefore a change in position of the driven fiber or fibers, which gives rise to a change in shape, in particular a swelling, of the implant as a result of the reduction in distance between the fiber ends, in particular essentially caused by the fact that the at least one driven fiber changes its profile or course or path or shape between the attachment points at the driving element from the direction of extent of the fiber which is present in the first shape into at least one direction which is perpendicular with respect thereto, in particular a radial direction.

In particular in this context “swelling” does not necessarily mean that the cross section of the driven fiber(s) is increased even though such an embodiment may be also possible. Particularly “swelling” means that the fiber (even if its cross section is maintained) occupies in an area between its respective ends in the second shape a bigger volume or at least cross section of the cavity compared to the first shape.

By means of this shortening, the implant brings about an enlargement of the occupied cross section perpendicular to the shortening direction, which automatically contributes to a filling of the body cavity. As a result of the preferred swelling of the at least one driven fiber, the filling density, that is to say the fiber content per volume unit of the body cavity, is automatically increased, the blood flow significantly reduced and the coagulation promoted. In particular, in a method for treating a body cavity by means of the automatic change in shape, a plurality of such implants can easily be successively positioned in a body cavity. It is a further advantage here that the user does not have to apply any plugging forces to the body cavity, since the filling of the cavity takes place automatically as a result of the change in shape.

In one preferred embodiment, the at least one driven fiber, in particular each driven fiber of a multiplicity of driven fibers, is attached by its respective ends to the ends of the at least one driving element which are spaced apart further in the first shape than in the second shape and preferably are spaced apart to a maximum degree. As a result, a change in shape can be achieved from a relatively large longitudinal extent, in particular a maximum one, starting in a direction of shortening. The degree of change in shape can therefore be maximized.

The invention can provide that the at least one driving element has, at its respective end, an attachment structure, to which in each case one of the ends of a respective driven fiber is attached. An attachment can be carried out e.g. by bonding or welding or by seals or press fixing. In the case of press fixing, the fixing, pressing force can be applied e.g. by the driving element itself.

An attachment structure can particularly preferably extend around the end, in particular in at least one direction perpendicular with respect to the longitudinal extent, preferably through 360 degrees in a plane perpendicular with respect to the longitudinal extent. This provides the possibility of arranging a plurality of driven fibers around the at least one driving element. The driven fibers can be arranged e.g. in the first shape in a plane in which the at least one driving element is also located, or the driven fibers can be arranged in three dimensions around the at least one driving element. For example, all or at least some of the fibers in the first shape can lie on an imaginary lateral surface of a cylinder. The fibers can therefore also fill a space arranged around a driving element.

In the first shape, the driven fibers preferably each have a linear extent between the attachment ends, but this is not absolutely necessary for the purpose of the invention. In the case of this linear extent, the fibers can be tensioned between the ends or the attachment structures of the at least one driving element. This can mean, in particular, that such a tensioned fiber is longer in this state than in a non-tensioned, that is to say relaxed, state. This lengthening of the fiber is preferably reversible. This abovementioned embodiment can preferably be combined with the texturing of a respective driven fiber or of a filament which is to be mentioned below.

The at least one driving element can be embodied e.g. by means of a single wire or multiple wire which has a straight longitudinal extent in the first shape or is arranged, e.g. wound or wrapped, e.g. helically wrapped, around a straight direction of longitudinal extent. The term “wire” does not necessarily imply an embodiment made of metal here, even if such an embodiment is preferred. In particular in the case of the wrapped embodiment, the preferred attachment variant mentioned at the beginning, in which the at least one driven fiber, preferably a multiplicity of fibers, are secured in the interior of the wrapping through pressing forces, e.g. by virtue of the fact that the fiber ends on both sides are inserted into the wrapping ends of the driving element, which are each likewise open on both sides, and are compressed by the wrappings, preferably with a compression force which is radial with respect to the central longitudinal axis.

The invention can also provide, in particular in conjunction with the abovementioned embodiment, that the at least one driving element is elastically deformed in the first shape with respect to the second shape. The driving element can therefore be deformed out of the second shape in the boundaries of its elastic deformability in the direction of the first shape, with the result that it automatically returns to the second shape if an influence which maintains the first shape, e.g. an external influence, such as for example by means of a catheter, is eliminated.

In one preferred embodiment, the at least one driving element, in particular a metallic element, can be deformed in a super elastic/pseudo-elastic fashion in the first shape, in particular can be present as a stress-induced martensite in the first shape and as an austenite in the second shape. Such an embodiment can be achieved e.g. by means of a metallic shape memory alloy, e.g. by using Nitinol as the material of the at least one driving element. The second shape can therefore be a “learnt” shape of the shape memory alloy, and the first shape can in turn be one which results from the second shape as a result of mere elastic deformation within the superplasticity limits. The first shape is therefore not intrinsically stable in this case either.

The invention can also provide for the at least one driving element to be constructed from a shape memory material and also to specifically use its shape memory, that is to say to change the shape between the first and the second by changing its temperature beyond a conversion temperature. For example, the change in temperature can take place as a result of contact with blood. In the first and second shape, the driving element is therefore intrinsically stable, i.e. retains the respective shapes without external influences, and the force which resets it from the first to the second shape is only caused by the change in temperature. In particular, in this way in both shapes the implant assumes a relaxed state in its respective temperature range which is assigned to the shape.

The invention can also provide in all possible embodiments that the at least one driven fiber does not have any further connection to the at least one driving element between the end-side attachment regions to the ends or attachment structures of the at least one driving element. In this case, a quasi-random change in shape of the at least one fiber can take place in the regions between the attachments by virtue of the change in shape.

The invention can also provide for the at least one fiber also to be attached in at least one region, preferably a plurality of regions, between the end-side attachments to the at least one driving element, e.g. in a direct contact-forming fashion or by means of an intermediate element, such as e.g. also a piece of fiber. As a result, the change in shape, preferably swelling in a predetermined region around the at least one driving element, can be restricted.

One preferred embodiment can provide in all embodiment variants that the at least one driven fiber is constructed as at least one textile filament. For example a polymer can be selected as material for such a textile filament, for example.

In a further preferred embodiment, the at least one textile filament can have a circumferential length per unit of cross-sectional area (that is to say considered perpendicularly with respect to the direction of extent) which is larger than in the case of a circular cross section, in particular has a cross section which deviates from the circular shape, can preferably comprise a grooved structure in the direction of extent. As a result, the surface of such a textile filament can be significantly increased, which has a further positive effect on the coagulation.

A fiber can e.g. be embodied in a trilobal fashion or in the shape of a snowflake in cross section. Likewise, a fiber can be configured in a hollow or porous fashion in all possible embodiments.

In a particularly preferred embodiment, the invention can provide that the at least one textile filament is formed from a filament which is textured in the relaxed state. As a result, the implant can be constructed in such a way that the at least one textured filament has, in the second shape of the driving element, a distance between its regions for attachment to the driving element which is smaller than the distance between the same regions in the relaxed state of the respective textured filament, in particular in comparison with the distance which the same ends of the filament would assume with respect to one another if the relaxed, textured filament were not attached to a driving element, and in this state is oriented in a relaxed and linear fashion. The texturing of the at least one textured filament in the first shape of the driving element is also preferably reduced, preferably removed by stretching.

In this specified embodiment, in the first shape of the at least one driving element, the driven textured textile filaments are therefore stretched, or tensioned, in comparison with their relaxed state, as a result of which the texturing is at least reduced, if appropriate is completely removed by the stretching.

In particular, the invention can provide that an implant with such stretched, textured filaments is kept in a dimensionally stable state by an inherently stable, driving element in the first shape. The invention can also provide that a state of the implant in which the textured filaments are stretched is present only if a force is applied to the implant by a pushing element within a catheter. For this purpose, the pushing element can be used to apply a pushing force to the end of the implant, which points to the implantation location, with the result that this end to which force is applied pulls back the other end.

These embodiments have the effect that a considerable increase in the surface area already results solely from the respective fiber when this fiber performs the change in shape, in particular when the texturing which is initially reduced or even removed by stretching returns again with the change in shape. Therefore, both texturing of the respective fiber and preferred swelling of the fibers occur with the change in shape.

According to the invention, the change in shape in the filaments can be carried out in two stages. If, in particular after the release from the catheter, the distance between the attachment ends of the driving element decreases, initially each stretched filament returns to a state in which it retains the texturing, in particular wherein it still has a longitudinal extent parallel to the longitudinal axis of the implant. Subsequently, the filaments change their shape together with the conversion of the driving element into its first shape. In particular, the filaments swell here, preferably as a result of them assuming a radially larger spatial region in comparison with the first shape. Texturing of a respective fiber can be constructed e.g. in an undulating shape, in particular with round and/or pointy undulations, for example as a knit/deknit texture and/or as false-twist texturing. Further preferred texturing types are e.g.: helical or twisted or darned or synfoam texture by means of an e.g. twist-untwist method or stretch core texture or lofted effect by texturing by means of an air jet, as well as high bulk texturing by means of stretch-relax methods.

Any inserted fiber can also be biodegradable, that is to say degrade over time in the body or be constructed as an e-spun or nano-fiber-based yarn.

In order to further promote thrombosis, the invention can also provide that the at least one driven fiber has a coating which acts in a coagulating fashion.

In order to facilitate the implantation, the implant can have, at the proximal implant end, a detachable connection to a guide element, e.g. a guide wire. Such a connection can be a connection which can be detached mechanically, electrolytically or electrothermally.

The invention can therefore provide that, in a first shape of the at least one driving element, the implant is pushed through a catheter with the guide element into a body cavity, released from the catheter into the body cavity and separated from the guide element, wherein or after which the at least one driving element changes from the first into the second shape, and as a result the at least one driven fiber simultaneously carries out a change in shape, in particular the entire implant consequently swells and more preferably the respective fiber textures.

Embodiments of the invention are described below with reference to the figures, in which:

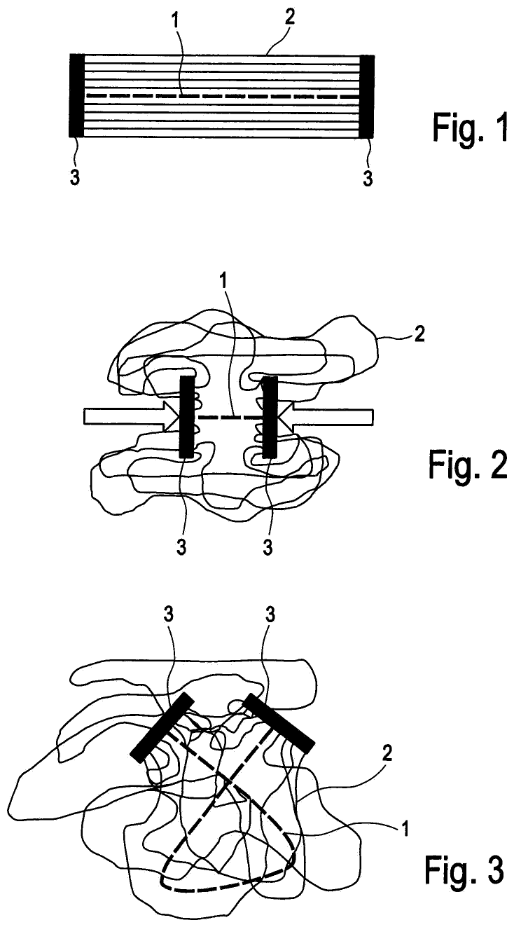

FIG. 1: shows an embodiment of an implant in a first shape of the driving element,

FIG. 2: shows the implant after an elastic change in shape of the driving element,

FIG. 3: shows the implant after a nonelastic change in shape of the driving element,

FIG. 4: shows the process of the pushing forward of an implant through a catheter,

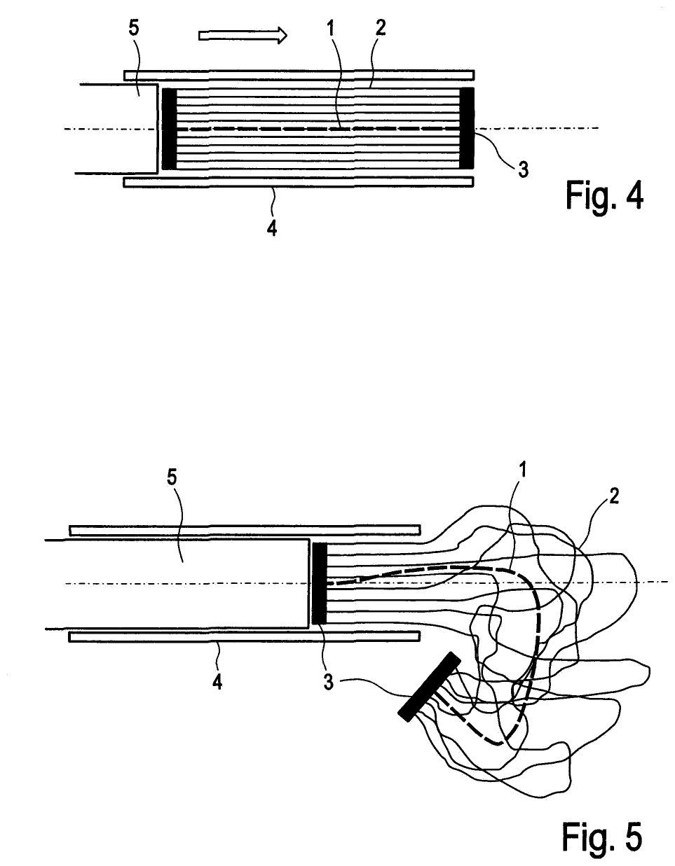

FIG. 5: shows the release of the implant from the catheter with immediate change in shape of the implant,

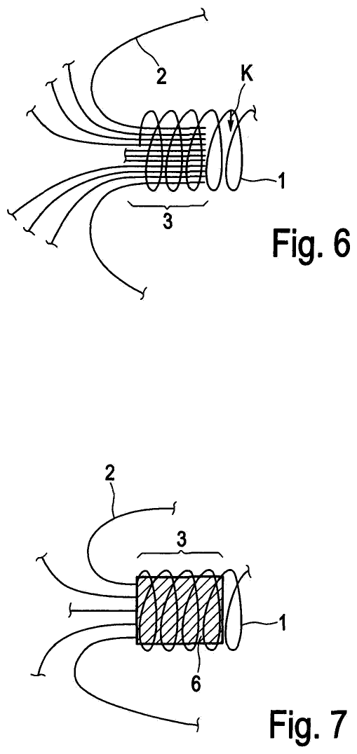

FIG. 6: shows an attachment possibility by means of pressing forces,

FIG. 7: shows an attachment possibility by means of a material join,

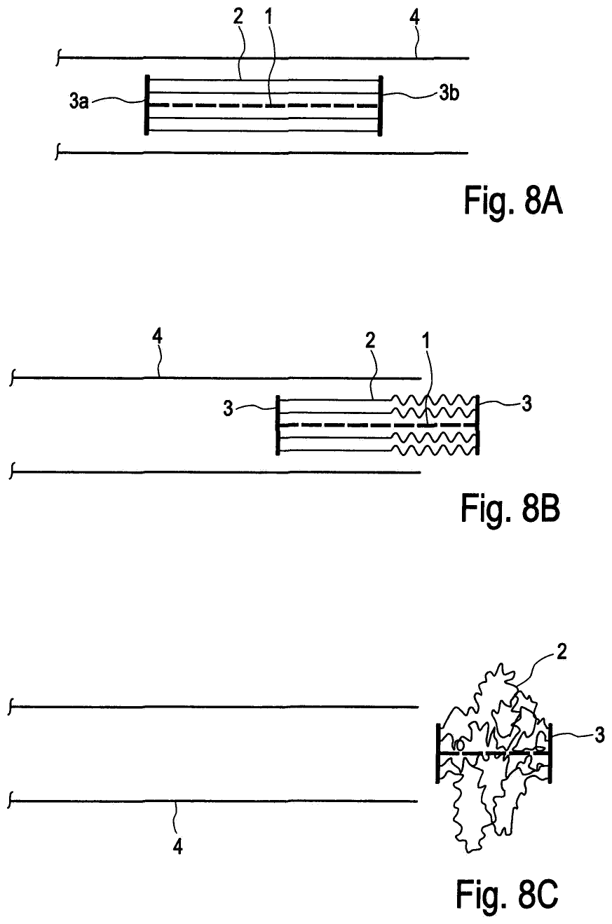

FIG. 8: shows an implantation process with textured filaments.

FIG. 1 shows a schematic view of the basic design of an implant according to the invention in a first shape which comes about when the driving element 1 is present in its first shape. In this embodiment, the driving element 1 has, at its ends which are spaced apart in the direction of extent, attachment structures 3 which extend here laterally, in particular perpendicularly, with respect to the direction of extent of the entire element 1. This results in the possibility of arranging a plurality of fibers 2 around the driving element. In particular, the driving element 1 can be located centrally between the fibers 2. The fibers are each attached by their ends to the attachment structures 3 lying opposite, and in the first shape of the element 1 are present in a likewise linearly extended shape.

FIG. 2 is a schematic view of a first possible action mechanism of the invention. Assuming that the element 1 has experienced elastic deformation in the first shape, owing to its elasticity the element 1 can return automatically to a second shape in which, according to FIG. 2, the element 1 is present in a shortened shape in comparison with the first shape. As a result of the shortening during the change in shape, the distance between the attachment structures 3 with respect to one another thus changes, which applies a shape-changing force to the fibers 2, which as a result change their relative position with respect to the element 1. In this context, the fibers also move out into regions which are located laterally beyond the ends of the attachment structures, with the result that swelling is produced by the shortening of the implant. A cavity in a body, for example an aneurysm, can in this way be filled well without there being the need to plug the implant into the cavity.

Owing to the shortening, which is purely elastic here, of the element 1, in this embodiment a shortening occurs in the direction of the spacing apart of the ends of the element. The element 1, which is extended in a linear fashion here, can be embodied e.g. as a helical spring. The elements 2 can then be fixed outside or inside the helical spring.

In contrast, FIG. 3 shows a possibility of a nonelastic change in shape, for which the driving element 1 is constructed e.g. from a shape memory material. The first shape of the element according to FIG. 1 can therefore be an intrinsically stable shape. As a result of contact with blood, heating can take place above a conversion temperature, which can bring about a change in shape into the shape shown in FIG. 3. It is basically to be noted that, in the first shape according to FIG. 3, stretching of an element 1 made of a shape memory material can also be present, which has taken place within the super-elasticity limits of the material, that is to say is not intrinsically stable. A second shape according to FIG. 3 can be applied to the material, which shape is intrinsically stable and into which the element 1 returns automatically if external shape-maintaining effects, e.g. as a result of a catheter, are eliminated.

It can be seen in FIG. 3 that, as a result of the change in shape, the position of the ends of the element 1 with respect to one another changes, in particular the distance is also reduced, as indicated in FIG. 2, but this change in distance does not take place along the connecting line of the ends of the element 1 in the first shape. As shown here, in the second shape the element 1 can have a shape which deviates from the linear extent, in particular a curved shape, in particular in which the element 1 can have a self-intersecting profile. As a result of the reduction in the distance which has also taken place here between the ends or attachment structures 3, swelling of the surrounding fibers occurs in turn.

The non-shaped-elastic element 1 can be embodied e.g. as a helical spring. The elements 2 can then be fixed outside or inside the helical spring.

FIG. 4 displays an implantation process. An implant according to FIG. 1 within a catheter 4 which is pushed forward through the catheter to the implantation location by means of a guide element 5 is illustrated. The implant is e.g. not deformed in an intrinsically stable fashion within the elasticity limit or within the super-elasticity limit of the element 1 and is held in shape by the catheter which is positioned around the outside of the implant.

FIG. 5 displays the moment of the emergence of the implant from the catheter 4, as a result of which the shape-maintaining effect of the catheter 4 is eliminated and the element 1 deforms immediately in the direction of its applied second intrinsically stable shape and in the process the fibers 2 swell.

FIG. 6 shows a possibility of attaching the individual driven fibers 2 to the attachment ends 3, shown on the left here, by means of a frictionally locking connection in the case of a helically wound, driving element 1, in particular one which corresponds approximately to a helical spring. On the right, the attachment is of identical design and the fibers 2 are guided from the left-hand side in an arcuate shape to the right-hand side. The free fiber ends are inserted here into the turns which are axially open at the end. The turns themselves apply a radial force K to the fiber ends, causing them to be held tight. In order to generate the radial force, there may be provision that, before the insertion of the fiber ends, the diameter of the turns is widened, with the result that the radial force is generated by the restoring force. FIG. 7 shows an alternative of a materially-joined connection. The ends of the fibers 2 at the attachment end 3 can also here be inserted axially into the open turns of the driving element 1 and are then bonded to the turns by means of a bonding agent 6.

FIGS. 8A to 8C clarify an implantation process of an implant with textured filaments as driven fibers 2.

FIG. 8A shows an implant in a catheter 4. The fibers 2 are formed by textured, textile filaments 2, the texturing of which is removed by stretching in the first shape of the driving element 1. The filaments 2 are therefore axially tensioned here.

This shape is brought about here by an axial, stretching force which is applied by a pushing element 5 which is guided past the attachment end 3a which points away from the implantation location and engages at the end 3b pointing towards the implantation location. The stretching is therefore present only during the pushing process.

There can alternatively also be provision that the stretched shape is maintained by an intrinsically stable, driving element. In this case, the embodiment is identical to FIG. 4.

FIG. 8B clarifies a partial emergence of the implant from the catheter 4. At least the emerged part of the filaments of the implant have retained their texturing, but the filaments are still longitudinally extended. If appropriate, the fibers/filaments end can already be structured again over their entire length.

FIG. 8C shows a case analogous to FIG. 5 in which in order to texture the filaments 2 the swollen shape of these filaments 2 now also occurs.

The driving element 1 has a shorter distance between its attachment ends 3 in the second shape than in the first shape, but in this embodiment is still axially linearly longitudinally extended. As an alternative, the element 1 can assume a shape as shown in FIG. 5 in which the distance between the attachment ends is reduced but the element 1 does not have a linearly extended profile.

1. An implant for insertion into body cavities, the implant comprising:
a driving element mechanically deformable from a second relaxed shape into a first deformed shape and reverting from the first deformed shape automatically into the second relaxed shape;
a longitudinally extending coagulation-inducing driven fiber having regions spaced longitudinally apart and attached to the at least one driving element in regions which are at a larger longitudinal spacing from one another in the first deformed shape than in the second relaxed shape, the driven fiber being moved by the driving element to change its shape with the conversion of the driving element from the first deformed shape into the second relaxed shape.

2. The implant according to claim 1, wherein the driven fiber has longitudinal ends attached to ends of the driving element that are spaced apart to a maximum degree in the first deformed shape.

3. The implant according to claim 2, wherein the driving element has at each of its respective ends an attachment structure to which each of the ends of a driven fiber is attached with the attachment structure extending around the respective end.

4. The implant according to claim 1, wherein the driving element is a wire having a straight longitudinal extent in the first deformed shape or is arranged around a straight direction of longitudinal extent.

5. The implant according to claim 1, wherein the driving element is elastically deformed in the first deformed shape.

6. The implant according to claim 1, wherein the driving element is deformed in a superelastic/pseudo-elastic fashion in the first deformed shape as a stress-induced martensite and as an austenite in the second relaxed shape.

7. The implant according to claim 1, wherein the driving element is constructed from a shape memory material and can be converted between the two shapes by changing its temperature past a conversion temperature.

8. The implant according to claim 1, wherein the driven fiber does not have any connection to the driving element between the attachment structures.

9. The implant according to claim 1, wherein the driven fiber is a textile filament.

10. The implant according to claim 9, wherein the textile filament has a circumferential length per unit of cross-sectional area which is larger than in the case of a circular cross section and has a cross section which deviates from the circular shape and is formed with a grooved structure in the direction of extent.

11. The implant according to claim 9, wherein the textile filament is formed from a filament which is textured in the relaxed state.

12. The implant according to claim 11, wherein the textured filament has in the second relaxed shape of the driving element a spacing between its regions for attachment to the driving element which is smaller than the distance between the same regions in the relaxed state of the respective textured filament.

13. The implant according to claim 11, wherein the texturing of the textured filament in the first deformed shape of the driving element is reduced by stretching.

14. The implant according to claim 1, wherein the driven fiber has a coating which acts in a coagulating fashion.

15. The implant according to claim 1, wherein the implant has at a proximal implant end, a detachable connection to a guide element that can be detached mechanically, electrolytically or electrothermally.

16. An implant comprising:
a pair of longitudinally spaced attachment structures;
an array of coagulation-promoting flexible filaments each having one end attached to one of the structures and another end attached to the other of the structures; and
a driving element extending between the structures and deformable between a first deformed state holding the structures apart at such a spacing that the filaments extend longitudinally straight between the structures a second relaxed state with the filaments loose and spreading transversely from between the structures.

17. The implant according to claim 16, wherein the driving element is elastically longitudinally compressible from the second state into the first state.

18. The implant according to claim 16, wherein the driving element is formed of shape-memory metal and shifts from the first state into the second state on being heated above a predetermined conversion temperature.
